(19) United States
(12) Patent Application Publication
(10) Pub. No.: US 2007/0077309 A1
(43) Pub. Date: Apr. 5, 2007

(54) Banded controlled release nanoparticle active agent formulation dosage forms and methods

(76) Inventors: Patrick S.L. Wong, Burlingame, CA (US); Andrew C. Lam, South San Francisco, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/527,103

(22) Filed: Sep. 25, 2006

Related U.S. Application Data
(60) Provisional application No. 60/723,134, filed on Sep. 30, 2005.

(51) Int. Cl. A61K 9/50; A61K 9/16

(52) U.S. Cl. 424/490

(57) ABSTRACT

Disclosed are controlled release dosage forms and related methods wherein controlled release of self-dispersing nanoparticle active agent formulations is provided by formulating porous particles into which have been sorbed a self-dispersing nanoparticle active agent formulation.

### CROSS REFERENCE TO RELATED U.S. APPLICATION DATA

The present application is derived from and claims priority to provisional application U.S. Ser. No. 60/723,134, filed Sep. 30, 2005, which is herein incorporated by reference in its entirety.

### FIELD OF THE INVENTION

This invention pertains to the controlled delivery of pharmaceutical agents and dosage forms therefor. In particular, the invention is directed to improved methods, dosage forms and devices for the controlled delivery of liquid active agent formulations to an environment of use.

### BACKGROUND OF THE INVENTION

The present inventors have previously taught and disclosed methods and devices, such as described in U.S. Pat. No. 6,342,249, incorporated herein by reference, for the controlled release of liquid, active agent formulations. The liquid, active agent formulations were loaded into porous particles that served as carriers for the liquid active agent formulations. The porous particles, loaded with liquid active agent formulations, could be formulated into osmotic, push-layer dosage forms. For certain drugs, the methods and devices taught in U.S. Pat. No. 6,342,249 do not provide optimal results and, in fact, present undesirable limitations, particularly in the aspect of dosage loading.

In past practice, administration of liquid active agent formulations was often preferred over solid active agent formulations in order to facilitate absorption of the active agent and obtain a beneficial effect for the intended use in the shortest possible time after the formulation is exposed to the environment of use. Examples of prior art devices to deliver liquid active agent formulations are soft gelatin capsules that contain a liquid active agent formulation or liquid formulations of the active agent that are bottled and dispensed in measured dosage amounts by the spoonful, or the like. Those systems are not generally amenable to controlled delivery of the active agent over time. While it is desired to have the active agent exhibit its effect as soon as it is released to the environment of use, it also often is desirable to have controlled release of the active agent to the environment of use over time. Such controlled release may be sustained delivery over time, such as zero order, or patterned delivery, such as pulsatile for example. Prior art systems have not generally been suitable for such delivery.

Various devices and methods have been described for the continuous delivery of active agents over time. Typically, such prior art systems have been used to deliver active agents initially in the dry state prior to administration. For example, U.S. Pat. Nos. 4,892,778 and 4,940,465, which are incorporated herein by reference, describe dispensers for delivering a beneficial agent to an environment of use that include a semipermeable wall defining a compartment containing a layer of expandable material that pushes a drug layer out of the compartment formed by the wall. The exit orifice in the device is substantially the same diameter as the inner diameter of the compartment formed by the wall.

U.S. Pat. No. 4,915,949, which is incorporated herein by reference, describes a dispenser for delivering a beneficial agent to an environment of use that includes a semipermeable wall containing a layer of expandable material that pushes a drug layer out of the compartment formed by the wall. The drug layer contains discrete tiny pills dispersed in a carrier. The exit orifice in the device is substantially the same diameter as the inner diameter of the compartment formed by the wall.

U.S. Pat. No. 5,126,142, which is incorporated herein by reference, describes a device for delivering an ionophore to livestock that includes a semipermeable housing in which a composition containing the ionophore and a carrier and an expandable hydrophilic layer is located, along with an additional element that imparts sufficient density to the device to retain it in the rumen-reticular sac of a ruminant animal. The ionophore and carrier are present in a dry state during storage and the composition changes to a dispensable, fluid-like state when it is in contact with the fluid environment of use. A number of different exit arrangements are described, including a plurality of holes in the end of the device and a single exit of varying diameter to control the amount of drug released per unit time due to diffusion and osmotic pumping.

It is often preferable that a large orifice, from about 50%-100% of the inner diameter of the drug compartment, be provided in the dispensing device containing the active agent and a bioerodible or degradable active agent carrier. When exposed to the environment of use, drug is released from the drug layer by erosion and diffusion. In those prior art instances where the drug is present in the solid state, the realization of the beneficial effect is delayed until the drug is dissolved in the fluids of the environment of use and absorbed by the tissues or mucosal environment of the gastrointestinal tract. For drugs that are poorly soluble in gastric or intestinal fluids, these delays found in the prior art are not preferred.

Devices in which the drug composition initially is dry but in the environment of use is delivered as a slurry, suspension or solution from a small exit orifice by the action of an expandable layer are described in U.S. Pat. Nos. 5,660,861, 5,633,011; 5,190,765; 5,252,338; 5,620,705; 4,931,285; 5,006,346; 5,024,842; and 5,160,743. Typical devices include an expandable push layer and a drug layer surrounded by a semipermeable membrane.

When the active agent is insoluble or poorly soluble, prior art systems may not provide rapid delivery of active agent or concentration gradients at the site of absorption that facilitate absorption through the gastrointestinal tract. Various approaches have been put forth to address such problems, including the use of water-soluble salts, polymorphic forms, powdered solutions, molecular complexes, micronization, eutectics, and solid solutions. An example of the use of a powdered solution is described by Sheth, et al., in “Use of Powdered Solutions to Improve the Dissolution Rate of Polythiazide Tablets,” Drug Development and Industrial Pharmacy, 16(5), 769-777 (1990). References to certain of the other approaches are cited therein. Additional examples of powdered solutions are described in U.S. Pat. 5,800,834. The patent describes methodology for calculating the amount of liquid that may be optimally sorbed into materials to prevent the drug solution from being exuded from the granular composition during compression.

U.S. Pat. No. 5,486,365, which is incorporated herein by reference, describes a spheronized material formed from a scale-like calcium hydrogen phosphate particulate material having a high specific surface area, good compressibility and low friability. That patent indicates that the material has the characteristic of high liquid absorption. However, the patent does not suggest that the material may be used as a carrier for delivery of a liquid medicament formulation to the environment of use. Instead, the patent describes the formation of a dried formulation, such as formed by spray drying. The patent describes the use of a suspension containing medicines and binders during the spray-drying granulation process to form a spherical particle containing the medicine. As an example, ascorbic acid in an amount equivalent to 10% of the scale-like calcium hydrogen phosphate was dissolved into a slurry of 20 weight percent of calcium hydrogen phosphate in water, and the resulting slurry was spray dried to form dried, spherical calcium hydrogen phosphate containing ascorbic acid. That material was then tableted under loads of 500-2000 kg/cm².

### SUMMARY OF THE INVENTION

In an aspect, the invention relates to a dosage form for the prolonged delivery of a self-dispersing nanoparticle active agent formulation absorbed in porous particles to a fluid environment of use comprising: a drug layer comprising a self-dispersing nanoparticle active agent formulation absorbed in porous particles, the porous particles being adapted to resist compaction forces sufficient to form a compacted drug layer without significant exudation of the self-dispersing nanoparticle active agent formulation; and at least two insoluble bands positioned in spaced relationship on a surface of the drug layer with the surface of the active agent formulation on both sides of each band exposed to the fluid environment of use.

In another aspect, the invention relates to a dosage form for the prolonged delivery of a self-dispersing nanoparticle active agent formulation absorbed in porous particles to a fluid environment of use comprising: a drug layer comprising a self-dispersing nanoparticle active agent formulation absorbed in porous particles; the porous particles having a mean particle size of ranging from about 50 to about 150 microns and being formed by spray drying a scale-like calcium hydrogen phosphate with a specific surface area of about 20 m²/g to about 60 m²/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:
CaHPO₄.mH₂O
wherein m satisfies the relationship O≦m≦2.0; and at least two insoluble bands positioned in spaced relationship on a surface of the drug layer with the surface of the active agent formulation on both sides of each band exposed to the fluid environment of use.

In another aspect, the invention relates to a dosage form for the prolonged delivery of a self-dispersing nanoparticle active agent formulation absorbed in porous particles to a fluid environment of use comprising: a drug layer comprising a self-dispersing nanoparticle active agent formulation absorbed in porous particles, the porous particles being calcium hydrogen phosphate having a specific volume of at least 1.5 ml/g, a BET specific surface area of at least 20 m²/g, and a water absorption capacity of at least 0.7 ml/g; and at least two insoluble bands positioned in spaced relationship on a surface of the drug layer with the surface of the active agent formulation on both sides of each band exposed to the fluid environment of use.

In yet another aspect, the invention relates to a dosage form for the prolonged delivery of a self-dispersing nanoparticle active agent formulation absorbed in porous particles to a fluid environment of use comprising: a drug layer comprising a self-dispersing nanoparticle active agent formulation absorbed in porous particles, the porous particles being calcium hydrogen phosphate having a specific volume of at least 1.5 ml/g, a BET specific area of at least 20 m²/g, and a water absorption capacity of at least 0.7 ml/g, the particles having a size distribution of 100% less than 40 mesh, 50%-100% less than 100 mesh and 10%-60% less than 200 mesh; and at least two insoluble bands positioned in spaced relationship on a surface of the drug layer with the surface of the active agent formulation on both sides of each band exposed to the fluid environment of use.

In yet another aspect, the invention relates to a dosage form for the prolonged delivery of a self-dispersing nanoparticle active agent formulation absorbed in porous particles to a fluid environment of use comprising: a drug layer comprising a self-dispersing nanoparticle active agent formulation absorbed in porous particles, the porous particles being calcium hydrogen phosphate having a bulk specific volume of 1.5 ml/g-5 ml/g, a BET specific area of 20 m²/g-60 m²/g, a water absorption capacity of at least 0.7 ml/g, and a mean particle size of at least 70 micrometers; and at least two insoluble bands positioned in spaced relationship on a surface of the drug layer with the surface of the active agent formulation on both sides of each band exposed to the fluid environment of use.

In yet another aspect, the invention relates to a method of facilitating the release of an active agent from a dosage form comprising: sorbing a self-dispersing nanoparticle active agent formulation of the active agent into and/or onto a plurality of porous particles, the particles, having a mean particle size of 50-150 microns, being formed by spray drying a scale-like calcium hydrogen phosphate with a specific surface area of 20 m²/g to 60 m²/g, an apparent specific volume of 1.5 ml/g or more, an oil absorption capacity of 0.7 ml/g or more, a primary particle size of 0.1μ to 5μ, and an average particle size of 2μto 10μ among secondary particles that are aggregates of the primary particles, the scale-like calcium hydrogen phosphate being represented by the following general formula:
CaHPO₄.mH₂O
wherein m satisfies the relationship O≦m≦2.0; and placing the self-dispersing nanoparticle active agent formulation of the active agent sorbed into and/or onto a plurality of porous particles into a fluid environment of use, said sorbed plurality of porous particles having at least two insoluble bands positioned in spaced relationship on a surface with the surface on both sides of each band exposed to the fluid environment of use; and allowing said sorbed plurality of porous particles to erode in the fluid environment of use while the bands remain positioned on the surface; wherein a surface area of said sorbed plurality of porous particles not covered by the bands increases with time.

In yet another aspect, the invention relates to a dosage form for the prolonged delivery of a self-dispersing nanoparticle active agent formulation absorbed in porous particles to a fluid environment of use comprising: a drug layer comprising a self-dispersing nanoparticle active agent formulation absorbed in porous particles, the porous particles being magnesium aluminometasilicate represented by the general formula
Al₂O₃MgO.2SiO₂.nH₂O
wherein n satisfies the relationship O≦n≦10; and at least two insoluble bands positioned in spaced relationship on a surface of the drug layer with the surface of the active agent formulation on both sides of each band exposed to the fluid environment of use.

Further, in another aspect, the invention relates to a dosage form for the prolonged delivery of a self-dispersing nanoparticle active agent formulation absorbed in porous particles to a fluid environment of use comprising: a drug layer comprising a self-dispersing nanoparticle active agent formulation absorbed in and/or onto porous particles, the porous particles being magnesium aluminometasilicate represented by the general formula
Al₂O₃MgO.2SiO₂.nH₂O
wherein n satisfies the relationship 0≦n≦10 and having a specific surface area of about 100-300 m²/g, an oil absorption capacity of about 1.3-3.4 ml/g, a mean particle size of about 1-2 microns, an angle of repose about 25°-45°, a specific gravity of about 2 g/ml and a specific volume of about 2.1-12 ml/g; and at least two insoluble bands positioned in spaced relationship on a surface of the drug layer with the surface of the active agent formulation on both sides of each band exposed to the fluid environment of use.

### BRIEF DESCRIPTION OF THE FIGURES

FIG. 1 illustrates a porous particle containing a self-dispersing active agent formulation according to the present invention;

FIG. 2 illustrates a composition comprising a plurality of particles containing a self-dispersing nanoparticle active agent formulation as illustrated in FIG. 1 dispersed in a carrier and suitable for use in dosage forms of the invention;

FIG. 3 is a side elevational view of one embodiment of the delivery device of the present invention, the device being in prepared form prior to placement in the environment of use.

FIG. 4 shows the device of FIG. 3 in operation after placement in the environment of use, showing erosion of the active agent formulation drug layer.

FIG. 5 shows the device of FIG. 3 in operation after sufficient erosion of the drug layer has caused separation of the banded sections of the device.

FIG. 6 is a side elevational view of a second embodiment of the delivery device of the present invention, the device being in prepared form prior to placement in the environment of use.

FIG. 7 shows the device of FIG. 6 in operation after sufficient erosion of the drug layer has caused separation of the sections of the device.

### DETAILED DESCRIPTION OF THE INVENTION

The present invention is best understood by reference to the following definitions, the drawings and exemplary disclosure provided herein.

I. DEFINITIONS

All documents cited herein are incorporated by reference in their entirety and for all purposes as if reproduced fully herein.

“Absorbed” or “sorbing” means being taken up by the volume.

“Active agent”, “drug”, or “compound” means an agent, drug, compound, composition of matter or mixture thereof which provides some physiological, psychological, biological, or pharmacological, and often beneficial, effect when in the environment of use.

“Apply” or “applied” or “application” means the substantially uniform deposition of insoluble material, in liquid or in molten form, onto the active agent formulation drug layer. A variety of techniques may be used to apply the insoluble material, including but not limited to Gravure-type printing, extrusion coating, screen coating, spraying, painting, and the Capsealer process developed by TAIT Design & Machine Co., Manheim, Pa.

“Active agent formulation” means the active agent or drug optionally in combination with pharmaceutically acceptable carriers and additional inert ingredients.

“Dosage form” means a pharmaceutical composition or device comprising an active pharmaceutical agent, the composition or device optionally containing inactive ingredients, such as pharmaceutically-acceptable carriers, excipients, suspension agents, surfactants, disintegrants, binders, diluents, lubricants, stabilizers, antioxidants, osmotic agents, colorants, plasticizers, and the like, that are used to manufacture and deliver active pharmaceutical agents.

“Drug layer” means a layer or volume that comprises an active agent formulation according to the invention.

“Facilitating the release of an active agent from a dosage form” means enhancing the efficiency or extent of release of an active agent from a dosage form according to the invention.

“Formulation loaded into the porous carrier” or “formulation loaded into the porous particles” means that the formulations are sorbed into, onto or otherwise mixed with the porous particles of the porous particle carrier.

“Insoluble” means a material that will not dissolve, degrade or erode in the environment of use during the delivery period.

“Liquid, active agent formulation” means that the active agent is present in a composition that is miscible with or dispersible in the fluids of the environment of use, or is able to flow or diffuse from the pores of the particles into the environment of use. The formulation may be neat, liquid active agent, or a solution, suspension, slurry, emulsion, self-emulsifying composition, colloidal dispersion or other flowable composition in which the active agent is present.

The active agent may be accompanied by a suspension agent, antioxidant, emulsion former, protecting agent, permeation enhancer and the like. The amount of an active agent in a dosage form generally is about 0.05 ng to 5 g or more, with individual dosage forms comprising, for example, 25 ng, 1 mg, 5 mg, 10 mg, 25 mg, 100 mg, 250 mg, 500 mg, 750 mg, 1.0 g, 1.2 g, and the like, of active agent. The system typically can be administered once, twice or thrice daily for pharmaceutical applications, or more or less as required by the particular application. In agricultural applications, systems typically will be applied at longer intervals, such as weekly, monthly, seasonally or the like.

“Nanoparticle” of drug means a drug particle having a mean particle size smaller than 2000 nm, more preferably a particle size ranging from about 30 to about 1500 n\grave{m}, more preferably about 100 to about 1000 nm, more preferably about 200 to about 600 nm. Additionally, the particles may preferably have a mean particle size of less than about 1500 nm, more preferably less than about 1000 nm, and more preferably less than about 600 nm.

“Pharmaceutically-acceptable acid addition salt” or “pharmaceutically-acceptable salt” means those salts in which the anion does not contribute significantly to the toxicity or pharmacological activity of the salt, and, as such, they are the pharmacological equivalents of the bases of the compounds to which they refer. Examples of pharmaceutically acceptable acids that are useful for the purposes of salt formation include but are not limited to hydrochloric, hydrobromic, hydroiodic, citric, acetic, benzoic, mandelic, fumaric, succinic, phosphoric, nitric, mucic, isethionic, palmitic, and others.

“Porous particle” means a plurality of porous particles or porous particulates of a homogenous or heterogenous composition. In an embodiment, the porous particles possess a mean particle size of ranging from about 50 to about 150 microns and being formed by spray drying a scale-like calcium hydrogen phosphate with a specific surface area of about 20 m²/g to about 60 m²/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:
CaHPO₄.mH₂O

wherein m satisfies the relationship O≦m≦2.0. In another embodiment, the porous particles comprise calcium hydrogen phosphate having a specific volume of at least 1.5 ml/g, a BET specific surface area of at least 20 m₂/g, and a water absorption capacity of at least 0.7 ml/g. In another embodiment, the porous particles have a bulk density of 0.4-0.6 g/ml, a BET surface area of 30-50 m₂/g, a specific volume of greater than 2 ml/g, and a mean pore size of at least 50 Angstroms. In an embodiment, the porous particles comprise calcium hydrogen phosphate having a specific volume of at least 1.5 ml/g, a BET specific area of at least 20 m²/g, and a water absorption capacity of at least 0.7 ml/g, the particles having a size distribution of 100% less than 40 mesh, 50%-100% less than 100 mesh and 10%-60% less than 200 mesh; more preferably the porous particles have a size distribution of 100% is less than 40 mesh, 60%-90% is less than 100 mesh and 20%-60% is less than 200 mesh. In an embodiment, the porous particles comprise calcium hydrogen phosphate having a bulk specific volume of 1.5 ml/g-5 ml/g, a BET specific area of 20 m₂/g-60 m²/g, a water absorption capacity of at least 0.7 ml/g, and a mean particle size of at least 70 micrometers. In an embodiment, the particles possess a mean particle size of 50-150 microns, being formed by spray drying a scale-like calcium hydrogen phosphate with a specific surface area of 20 m²/g to 60 m₂/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:

ti CaHPO₄.mH₂O

wherein m satisfies the relationship O≦m≦2.0. In an embodiment, the porous particles comprise magnesium aluminometasilicate represented by the general formula
Al₂O₃MgO.2SiO₂.nH₂O

wherein n satisfies the relationship O≦n≦10. In an embodiment, the porous particles comprise magnesium aluminometasilicate represented by the general formula
Al₂O₃MgO.2SiO₂.nH₂O

wherein n satisfies the relationship O≦n≦10 and having a specific surface area of about 100-300 m₂/g, an oil absorption capacity of about 1.3-3.4 ml/g, a mean particle size of about 1-2 microns, an angle of repose about 25°-45°, a specific gravity of about 2 g/ml and a specific volume of about 2.1-12 ml/g.

“Prolonged period of time” or “prolonged period” means a continuous period of time of 4 hours or more, more typically 6 hours or more. The phrase “prolonged delivery” intends a period of delivery that lasts for several hours to about 24 hours, usually up to about 20 hours, and often between about 3 and 16 hours.

“Pulsatile release” means release of an active agent to an environment of use for one or more discrete periods of time preceded or followed by (i) at least one discrete period of time in which the active agent is not released, or (ii) at least one period of time in which another, different active agent is released. Pulsatile release is meant to include delayed release of active agent following administration of the dosage form and release in which one or more pulses of active agent are released over a period of time.

“Release rate assay” means a standardized release assay for the determination of a compound using a USP Type 7 interval release apparatus substantially in accordance with the description of the assay contained herein. It is understood that reagents of equivalent grade may be substituted in the assay in accordance with generally-accepted procedures. Also, different fluids such as artificial gastric fluid or artificial intestinal fluid may be used to evaluate release characteristics in environments characterized by different pH values. “Release” and “release rate” are determined by a release rate assay.

“Self-dispersing nanoparticle active agent formulation” means a liquid active agent formulation which comprises nanoparticles of active agent and which can disperse in an aqueous medium without vigorous agitation. Some of the active agent in a self-dispersing nanoparticle active agent formulation may also be dissolved in the liquid active agent formulation. The formulation serves to disperse in the gastrointestinal environment and can provide emulsion vehicles or emulsion bodies that distribute the nanoparticles in the gastrointestinal environment and also facilitate enhanced dissolution of the active agent in the gastrointestinal environment.

“Steady state” means the condition in which the amount of drug present in the blood plasma of a subject does not vary significantly over a prolonged period of time.

“Sustained release” means continuous release of active agent to an environment of use over a prolonged period.

“Therapeutically effective” amount or rate means the amount or rate of the active agent needed to effect the desired pharmacologic, often beneficial, result.

“Uniform rate of release” or “uniform release rate” means a rate of release of the active agent from a dosage form that does not vary positively or negatively by more than 30% from the mean rate of release of the active agent over a prolonged period of time, as determined in a USP Type 7 Interval Release Apparatus. Preferred uniform rates of release will vary by not more than 25% (positively or negatively) from the mean rate of release determined over a prolonged period of time.

II. Introduction

It has been found that various beneficial effects are gained by the use of drug nanoparticles as active agents in a self-dispersing nanoparticle active agent formulation loaded into and/or onto porous particles. This is particularly true for drugs that exhibit low solubility in the gastrointestinal environment such as Class II and Class IV drugs as defined by the U.S. FDA Biopharmaceutical Classification System.

The present invention provides for improved delivery characteristics of the nanoparticle formulation. The inventors have unexpectedly realized that certain dosage forms that may comprise self-dispersing nanoparticle active agent formulation absorbed onto porous particles may not effectively deliver the porous particle formulation efficiently into an environment of use. As a particular advantage, the dosage forms of the present invention provide for less residual material being left in the dosage form following dosing as compared to the prior art. Without wishing to be bound to a particular mechanism, the inventors believe that the inventive dosage forms provide for enhanced water flux into the self-dispersing nanoparticle active agent formulation absorbed onto porous particles as compared to prior art dosage forms. This enhanced water flux may provide increased levels of disintegration of the porous particles, thus providing improved efficiency of delivery. Improved efficiency of delivery can result in smaller dosage form sizes, meaning that the inventive dosage forms are easier to swallow, and can reduce the cost of the dosage form.

Additionally, the nanoparticle formulation of the present invention provides an advantage because the absorbed nanoparticles are less likely to agglomerate or settle within the small pore spaces in the porous particles. Also growth of the nanoparticles is limited due to the limited amount of soluble drug within the pores of the porous particles (i.e. Oswald ripening is inhibited).

These advantages and others will now be described in more detail below.

Ill. Nanoparticles and Formulations

In some embodiments of the present invention the self-dispersing nanoparticle formulation is in the form of emulsions or self-emulsifying compositions as defined herein. Due to the increased solubility of the drug provided by the self-emulsifying composition, creation of relatively higher concentrations of dissolved drug in the gastrointestinal tract are achieved. Moreover, because the emulsion works to solubilize the drug in the gastrointestinal environment as the already dissolved drug material is absorbed by the body, the self-emulsifying suspension works to maintain a higher concentration of dissolved drug in the gastrointestinal tract over a longer period of time than would be possible if the formulation simply included an amount of the dissolved drug. This, then, leads to preferred faster and greater absorption of the drug. These improvements are in addition to the improvements due to improved delivery from the inventive dosage forms.

In certain preferred embodiments, the self-dispersing nanoparticle formulation is one in which the formulation, when released from a dosage form in the gastrointestinal tract, can disperse in the aqueous media of the gastrointestinal tract without vigorous agitation, or in other words, can disperse in the aqueous media of the gastrointestinal tract by effect of the motility of the gastrointestinal tract.

Some of the benefits of the present invention arise from characteristics of the nanoparticles themselves. Nanoparticles of a drug dissolve more quickly than larger sized particles of the same drug. One reason is that, since geometrically an equal weight of nanoparticles has a greater surface area than does an equal weight of larger particles of the same drug, a nanoparticle form of a drug has a greater surface area available for dissolution of the drug from the drug particles or crystals than does an equal weight of the drug in a form composed of larger sized particles. Additionally, nanoparticles inherently have a more irregular surface area and crystal structure than do larger more regular drug crystals. Since dissolution from the irregular surface crystal structure of nanoparticles occurs more readily than from a regular crystal surface and structure of larger sized particles, nanoparticles dissolve more readily than do larger particles of the same drug.

If nanoparticles are simply packed into a drug form without the other aspects of the present invention, the drug particles or crystals tend to combine or agglomerate. The resultant larger drug particles of the drug form undesirably dissolve more slowly in the gastrointestinal tract than do non-agglomerated nanoparticles of the drug.

By mixing the drug nanoparticles into a self-dispersing carrier and then loading the resulting self-dispersing nanoparticle formulation into porous particle carriers, undesired growth or agglomeration of drug particles is inhibited. When drug nanoparticles are mixed into a self-dispersing carrier without then loading the mixture into porous particle carriers, it is typical that the nanoparticles, or at least usually the larger nanoparticles, will grow by the phenomenon of Oswald ripening. However, when such a mixture is loaded into porous particle carriers the porous particles tend to provide, in many of the preferred formulations, a physical separation between the nanoparticles (and, as explained below, between portions of the liquid carrier) and will minimize or eliminate Oswald ripening growth of a substantial portion of the nanoparticles. It should be understood that the porous particles, by capillary and other actions, absorb the bulk of the liquid carrier and thus provide a physical separation between the nanoparticles and also virtually eliminate liquid communication between the nanoparticles. This eliminates or largely prevents Oswald ripening induced growth of the nanoparticles. This presents obvious advantages over systems in which the nanoparticles are packed together in a dosage form and then tend to agglomerate.

Clearly, these aspects of the present invention present beneficial advantages over systems in which nanoparticles are provided in suspension (without loading into porous carriers) wherein the nanoparticles of such systems frequently grow, agglomerate or combine during storage in the suspension formulation. This growth or agglomeration diminishes the solubility of the drug and effectiveness of the drug forms in which the drug is embodied. Additionally, such suspensions of nanoparticles cannot be handled with dosage form manufacturing equipment designed to process dry constituents of dosage forms, while self-dispersing nanoparticle formulations sorbed into porous particle carriers according to the present invention can be processed by such equipment.

Thus, the present invention may improve stability of the nanoparticles in the self-dispersing nanoparticle active agent formulation, while still providing relatively easy dispersion upon exposure to the fluid environment of use. This may result in improved bioavailability.

According to various aspects or embodiments of the present invention, the self-dispersing nanoparticle active agent formulation can be sorbed into the pores of the porous particle carrier. Additionally, nanoparticles of the formulation can adhere to the outside of the porous particle for reasons such as the wetness of the surface of the porous particle effected by the self-dispersing formulation.

The present invention achieves the combined objectives of a high drug loading while maintaining and without compromising high dissolution characteristics.

Nanoparticles used in the present invention preferably have a mean particle size less than 2000 nm, more preferably they range from 20 to 2000 nm, more preferably 30 to 1500 nm, even more preferably 100 to 1000 nm, more preferably 200 to 600 nm. Additionally, the particles may preferably have a mean particle size of less than 1500 nm, more preferably less than 1000 nm, and more preferably less than 600 nm.

Nanoparticles of drugs for use according to embodiments of the present invention can be prepared using any process providing particles within a desired range of sizes. For example, the drug may be processed using a wetmilling or supercritical fluid process, such as an RESS or GAS process. In addition, processes for producing nanoparticles are disclosed in U.S. Pat. Nos. 6,267,989, 5,510,118, 5,494,683, and 5,145,684. Nanoparticles may also be formed according to methods described elsewhere herein for the formation of drug particles.

In the use of nanoparticles according to some embodiments of the present invention, it is useful to process the drug or nanoparticles of drug with one or more coating agents to minimize particle aggregation or agglomeration. Exemplary coating agents include lipids, hydrophilic polymers, such as hydroxypropyl methylcellulose (“HPMC”) and polyvinylpyrrolidone (“PVP”) polymers, and solid or liquid surfactants. The coating agent used in a nanoparticle forming process may also include a mixture of agents, such as a mixture of two different surfactants. Where used as a coating agent, a hydrophilic polymer may work to both facilitate formation of nanoparticulate material and stabilized the resulting nanoparticles against recrystalization over long periods of storage. Surfactants useful as coating agents in the creation of nanoparticles useful in the self-emulsifying nanosuspension of the present invention include nonionic surfactants, such as Pluronic F68, F108, or F127. the non-ionic surfactants already mentioned herein may also be useful as coating agents in a nanoparticle forming process.

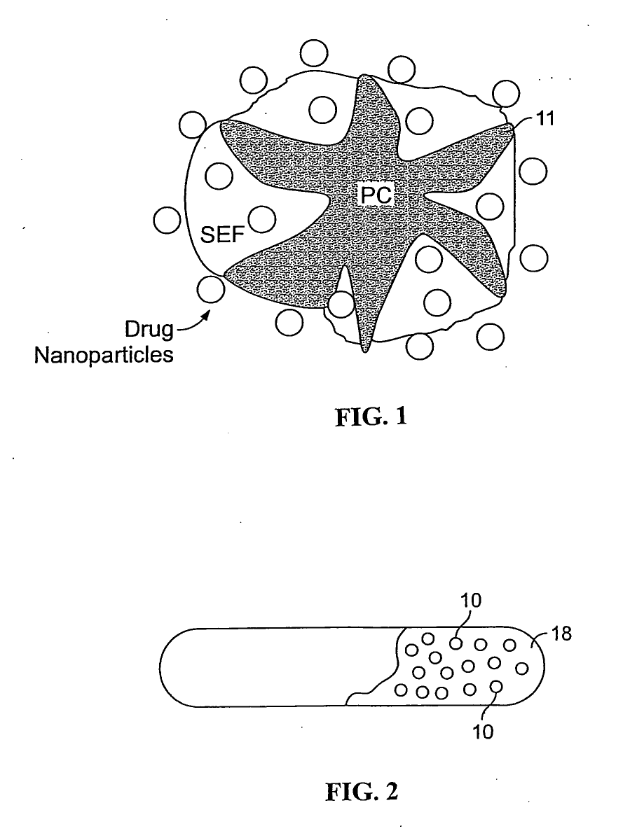

FIG. 1 illustrates a porous particle 10 having a material mass 11 that defines a plurality of pores 12 and which has been loaded with a self-dispersing nanoparticle formulation 14 comprising a self-dispersing liquid carrier and active agent nanoparticles 16. Within pores 12 is sorbed the self-dispersing formulation 14. Nanoparticles 6 are not only contained in the pores 12 but also can adhere to the outside of the porous particle 10 due to factors such as potential wetness of the surface of porous particle 10 effected by the self-dispersing formulation 14. Pores 14 extend from the external surface of the particle and into the interior. Pores are open on the surface to permit the self-dispersing nanoparticle active agent formulation to be sorbed into the particles by conventional mixing techniques such as wet granulation, spraying of the self-dispersing nanoparticle active agent formulation onto a fluidized bed of the particles, or the like. Additionally, according to embodiments of the present invention, some percentage of the drug may be dissolved in the liquid carrier.

When manufacturing the inventive dosage forms, a common practice may be to fabricate a compressed tablet comprising the drug layer and the push layer. The drug layer composition may be conveniently compressed in granulated or powdered form in a die cavity of a tabletting press. During the compression or compacting step of the drug layer, the porous particles should be sufficiently resistant to the compressive forces so as not to be crushed or pulverized to any significant extent and prematurely release the self-dispersing nanoparticle active agent formulation from the porous particles.

Materials useful for sorbing the self-dispersing nanoparticle active agent formulations are porous particulates that are characterized by high compressibility or tensile strength to withstand compacting forces applied during compacting steps and minimize exudation of self-dispersing nanoparticleself-dispersing nanoparticle active agent formulation from the pores; particle flow characteristics that allow for the porous particles to be directly compacted without the use of a binder or with minimal use of a binder; low friability so as to preclude or minimize exudation of the liquid and facilitate tablet cohesion, active agent formulation from the particles during compacting steps; and high porosity so as to absorb an adequate of amount of a self-dispersing nanoparticle active agent formulation to provide an effective amount of active agent in a dosage form. The particles should be adapted to absorb an amount of self-dispersing nanoparticles active agent formulation such that a therapeutically effective amount of the active agent may be delivered in a unitary dosage form that is of a size that can be conveniently swallowed by a subject and, preferably provided in four or fewer tablets or capsules for ingestion at the same time. The porosity of the particles may be such that at least 5% and up to 70%, more often 20-70%, preferably 30-60%, and more preferably 40-60%, by weight of the self-dispersing nanoparticle active agent formulation, based on weight of the particles may be sorbed into the pores of the particles, while the particles exhibit sufficient strength at such degree of active agent loading so as not to significantly be crushed or pulverized by compacting forces to which the particles will be subjected during manufacturing operations. More typically, the self-dispersing nanoparticle active agent formulation may comprise 30-40% of the weight of the porous particles when the particles are crystalline, such as calcium hydrogen phosphate, but that percentage may be greater, e.g., up to 60-70% or more when more amorphous materials, such as magnesium aluminometasilicates, are used. Blends of crystalline and amorphous material may be utilized. At high loadings, it may be advantageous to use blends of calcium hydrogen phosphate particles and amorphous magnesium aluminometasilicate powders.

Preferred materials are those having a strength to resist compression forces of greater than 1500 kg/cm² without substantial exudation of the self-dispersing nanoparticle active agent formulation, and most preferably without the tablet hardness plateauing.

A particularly suitable porous particle is exemplified by the particular form of calcium hydrogen phosphate described in U.S. Pat. No. 5,486,365, which is incorporated herein by reference. As described therein, calcium hydrogen phosphate is prepared by a process yielding a scale-like calcium hydrogen phosphate that can be represented by the formula CaHPO₄.mH₂O wherein m satisfies the expression 0 ≦m ≦0.5. Useful calcium hydrogen phosphate materials may include those of the formula CaHPO₄.mH₂O wherein m satisfies the expression 0 ≦m ≦2.0. The scale-like calcium hydrogen phosphate produced has characteristic physical properties that make it particularly suitable for use in the present invention. The scale-like material provides high specific surface area, high specific volume, high capacity for water and oil absorption, and the ability to readily form into spheres upon spray drying. The spherical particulates have excellent flow properties and permit direct compaction into tablets without binders and without significant crushing or pulverizing of the particles during the compaction step.

The scale-like calcium hydrogen phosphate particles generally have a BET specific surface area of at least 20 m²/g, typically 20 m²/g -60 m ₂/g, a specific volume of at least 1.5 ml/g, typically 2-5 ml/g or more, and an oil and water absorption capacity of at least 0.7 ml/g, typically 0.8-1.5 ml/g. When formed into spheres the spherical particulates may have a mean particle size of 50 microns or greater, usually about 50-150 microns, and often about 60-120 microns. The particle size distribution may be 100% through 40 mesh, 50%-100% through 100 mesh, and 20%-60% through 200 mesh. The bulk density may be from about 0.4 g/ml-0.6 g/ml.

A most preferred form of calcium hydrogen phosphate is that sold under the trademark FujiCalin® by Fuji Chemical Industries (U.S.A.) Inc., Robbinsville, N.J., in types SG and S. Typical parameters for that material include a mean particle size of 500-150 microns, a mean pore size on the order of 70 Angstroms, a specific volume of about 2 ml/g, a BET specific surface area of about 30-40 m²/g, and an oil and water absorption capacity of about 0.7 ml/g. Type SG typically will have a mean particle size of about 113 microns, and a particle size distribution of 100% through 40 mesh, 60% through 100 mesh and 20 through 200 mesh. Type S typically will have a mean particle size of about 68 microns, and a particle size distribution of 100% through 40 mesh, 90% through 100 mesh and 60% through 200 mesh. Mixtures of the two types may be conveniently employed to provide particulates having physical characteristics that are suitable for various applications, as may be determined by those skilled in the art of pharmaceutical formulation, tableting and manufacturing.

The calcium hydrogen phosphate has low friability, demonstrating a tensile strength of up to about 130 kg/cm² when subjected to compressive forces of up to 3000 kg/cm². The hardness of the tableted material tends not to plateau at compression forces to that limit, while materials such as microcrystalline cellulose (Avicel PH 301), lactose, Dl-TAB and Kyowa GS tend to plateau at or about 700-1500 Kg/cm². The angle of repose for the preferred materials typically is on the order of 32-35 degrees.

Another material that may be utilized is that formed of magnesium aluminometasilicate which may be represented by the general formula
Al₂O₃MgO.2SiO₂.nH₂O

wherein n satisfies the relationship 0≦n≦1 0. Commercially available magnesium aluminometasilicates are sold as Grades S₁, SG₁, UFL₂, US₂, FH₁, FH₂, FL₁, FL₂, S₂, SG₂, NFL₂N, and NS₂N, under the trademark Neusilin™ by Fuji Chemical Industries (U.S.A.) Inc., Robbinsville, N.J. Especially preferred grades are S₁, SG₁, US₂ and UFL₂, with US₂ presently being most preferred. Those materials which are amorphous typically have a specific surface area (arca) of about 100-300 m²/g, an oil absorption capacity of about 1.3-3.4 ml/g, a mean particle size of about 1-2 microns, an angle of repose about 25°-45°, a specific gravity of about 2 g/ml and a specific volume of about 2.1-12 ml/g.

Other absorptive materials may be substituted for the foregoing or blended therewith, such as for example, powders of microcrystalline cellulose sold under the tradenames Avicel (FMC Corporation) and Elcema (Degussa); porous sodium carboxymethyl cellulose crosslinked sold as Ac-Di-Sol (FMC Corporation); porous soy bean hull fiber sold under the tradename Fl-1 Soy Fiber (Fibred Group); and porous agglomerated silicon dioxide, sold under the tradenames Cab-O-Sil (Cabot) and Aerosil (Degussa). Further materials are disclosed in published patent application 2005/0181049 to Dong et al.

The self-dispersing nanoparticle active agent formulation may be in any form that can be dispensed from the porous particles as the drug layer disintegrates in the environment of use. Optionally other dosage-forming ingredients, such as an anti-oxidant, a suspending agent, a surface active agent, and the like may be present in the self-dispersing nanoparticle active agent formulation. The self-dispersing nanoparticle active agent formulation will be released in a form most suitable to provide active agent to the site of delivery in a state in which it may be rapidly dissolved and absorbed in the environment of use to provide its beneficial action with minimum delay once delivered to the absorption site.

IV. Dosage Forms

The dosage forms of the invention find use, for example, in humans or other animals. The environment of use is typically a fluid environment and can comprise the stomach, or the gastrointestinal intestinal tract. A single dispensing device or several dispensing devices can be administered to a subject during a therapeutic program.

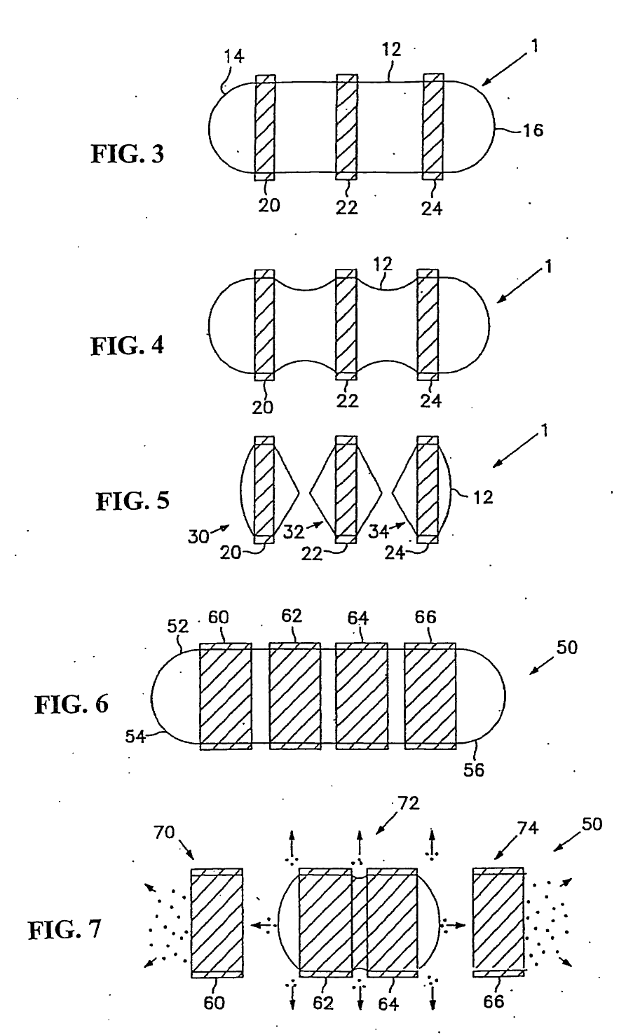

FIG. 3 depicts, in side elevational view, one embodiment of the dosage form according to the present invention. The dosage form is shown in prepared form prior to placement in the environment of use. Dosage form 1 is shown in FIG. 3 to comprise a cylindrically shaped drug layer 12, which comprises a self-dispersing nanoparticle active agent formulation adsorbed onto porous particles. The ends 14 and 16 of the drug layer are preferably rounded and convex in shape in order to ensure ease of insertion into the environment of use. Bands 20, 22 and 24 concentrically surround the cylindrical drug layer 12.

FIG. 4 shows dosage form 1 in operation after having been placed in the fluid environment of use. The active agent formulation drug layer 12 between bands 20, 22 and 24 has begun to erode, thereby releasing active agent to the fluid environment of use.

FIG. 5 shows dosage form 1 in operation after a length of time in the fluid environment of use. The active agent formulation drug layer 12 has eroded between bands 20, 22 and 24 to such an extent that the drug layer 12 is now in three pieces, 30, 32 and 34. Erosion will continue until the drug layer portions of each of the pieces have completely eroded. Bands 20, 22 and 24 will thereafter be expelled from the fluid environment of use.

FIG. 6 shows, in side elevational view, a second embodiment of the dosage form according to the present invention. The dosage form is shown in prepared form prior to placement in the environment of use. Dosage form 50 comprises a cylindrically shaped active agent formulation drug layer 52 with convex ends 54 and 56. Bands 60, 62, 64 and 66 concentrically surround the cylindrical drug layer 52.

FIG. 7 shows dosage form 50 in operation after a length of time in the fluid environment of use. The active agent formulation drug layer 52 has eroded from the exposed ends of bands 60, and 66 to such an extent that the dosage form 50 is now in three pieces, 70, 72, and 74. The arrows show the erosion of the drug layer and therefore the extent of active agent delivery. Erosion will continue from the exposed ends of bands 62 and 64 until the drug layer has completely eroded. Bands 60, 62, 64 and 66 will thereafter be expelled from the fluid environment of use.

The amount of active agent employed in the dosage form will be that amount necessary to deliver a therapeutically effective amount of the agent to achieve the desired result at the site of delivery. In practice, this will vary widely depending upon the particular agent, the site of delivery, the severity of the condition, and the desired therapeutic effect. Thus, it is not practical to define a particular range for the therapeutically effective amount of active agent incorporated into the device.

The hydrophilic polymeric material useful herein may comprise, polysaccharides, methyl cellulose, sodium or calcium carboxymethyl cellulose, hydroxypropylmethyl cellulose, hydroxypropyl cellulose, hydroxyethyl cellulose, nitrocellulose, carboxymethyl cellulose and other cellulose ethers, and polyethylene oxides (eg, Polyox.®™, Union Carbide). Other materials useful as the hydrophilic polymeric material include but are not limited to methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (Eudragit.™) or other acrylic acid derivatives, sorbitan esters, natural gums, lecithins, pectin, alginates, ammonia alginate, sodium or potassium alginate, calcium alginate, propylene glycol alginate, potassium alginate, agar, gum arabic, gum karaya, locust bean gum, gum tragacanth, carrageenans, gum ghatti, guar gum, xanthan gum, scleroglucan, and blends of the above.

In addition to design of the active agent formulation to provide a specific drug delivery profile, the number, size, and placement of the insoluble bands that are applied onto the active agent formulation drug layer may be varied to provide the desired drug delivery profile. For example, bands of from about 0.1 mm to about 12 mm in width, preferably between about 0.5 and 8 mm, may be applied onto the active agent formulation drug layer surface. Further, between about 2 and 10 bands may be used, but generally between about 2 and 6 are affixed to the drug layer. The bands may be placed close together (ie, within about 0.5 mm of each other) or may be placed at opposite ends of the drug layer (ie, spaced about 8 to 12 mm apart). The insoluble material may be any material that is nontoxic, biologically inert, nonallergenic and nonirritating to body tissue, and that maintains its physical and chemical integrity; that is, the bands do not erode or degrade in the environment of use during the dispensing period. Insoluble materials from which the bands may be prepared include, for example, polyethylene, polystyrene, ethylene-vinyl acetate copolymers, polycaprolactone and Hytrel.®™ polyester elastomers (Du Pont). Additional banding materials include but are not limited to polysaccharides, cellulosics, powdered cellulose, microcrystalline cellulose, cellulose acetate, cellulose actetate pseudolatex (such as described in U.S. Pat. No. 5,024,842), cellulose acetate propionate, cellulose acetate butyrate, ethyl cellulose, ethyl cellulose pseudolatex (such as Surelease.®™ as supplied by Colorcon, West Point, Pa. or Aquacoat.™ as supplied by FMC Corporation, Philadelphia, Pa.), dispersing nitrocellulose, polylactic acid, poly-glycolic acid, polylactide glycolide copolymers, collagen, polycaprolactone, polyvinyl alcohol, polyvinyl acetate, polyethylene vinylacetate, polyethylene teraphthalate, polybutadiene styrene, polyisobutylene, polyisobutylene isoprene copolymer, polyvinyl chloride, polyvinylidene chloride-vinyl chloride copolymer, copolymers of acrylic acid and methacrylic acid esters, copolymers of methylmethacrylate and ethylacrylate, latex of acrylate esters (such as Eudragit.®™ supplied by RohmPharma, Weiterstadt, Germany), polypropylene, copolymers of propylene oxide and ethylene oxide, propylene oxide ethylene oxide block copolymers, ethylenevinyl alcohol copolymer, poly sulfone, ethylene vinylalcohol copolymer, polyxylylenes, polyamides, natural and synthetic waxes, paraffin, carnauba wax, petroleum wax, white or yellow bees wax, castor wax, candelilla wax, rice bran wax, microcrystalline wax, stearyl alcohol, cetyl alcohol, bleached shellac, esterified shellac, chitin, chitosan, silicas, polyalkoxysilanes, polydimethyl siloxane, polyethylene glycol-silicone elastomers, crosslinked gelatin, zein, electromagnetic irradiation crosslinked acrylics, silicones, or polyesters, thermally crosslinked acrylics, silicones, or polyesters, butadiene-styrene rubber, glycerol ester of partially dimerized rosin, glycerol ester of partially hydrogenated wood rosin, glycerol ester of tall oil rosin, glycerol ester of wood rosin, pentaerythritol ester of partially hydrogenated wood rosin, pentaerythritol ester of wood rosin, natural or synthetic terpene resin and blends of the above.

The banding materials often are also formulated with plasticizers, and optionally with wetting agents, surfactants, opacifiers, colorants, flavorants, taste-masking agents, and the like. Examples of typical plasticizers are as follows: polyhydric alcohols, polyethylene glycol, glycerol, propylene glycol, acetate esters, glycerol triacetate, triethyl citrate, acetyl triethyl citrate, glycerides, acetylated monoglycerides, oils, mineral oil, castor oil and the like.

The rate of release of the active agent from the inventive dosage form is predominantly controlled by erosion of the hydrophilic gel formed by contacting the drug layer with the fluid environment of use. The drug released, m at time t, is proportional to the surface area of the system and can be written as
dm/dt=KA. (Equation 1)

K is the erosion constant in mg/cm² hr and varies according to the molecular weight, particle size and hydrophilicity of the drug layer, the solubility of the drug in the environmental use, and the hydrodynamic condition of the erosive media.
A is the erosion area.
The release profile dm/dt is constant if K and A remain constant.

Substituting Equation 1 with the area of a cylindrical drug layer with negligible end effects gives:
dm/dt=K2πRL (Equation 2)
where L is the length of the cylinder and
R is the radius of the cylinder at time t.
The mass release at time t is
   m=π[R₀²−R²]LC₀ (Equation 3)
R₀ is the initial radius of the cylinder and
C₀ is the initial concentration of the drug in the cylinder.

Substituting Equation 3 into Equation 2 leads to
dR/dt=−K/C₀ (Equation 4)

Integrating Equation 4 gives
R=R₀−(K/C₀)t (Equation 5)
The fraction amount of drug release, F, can now be defined by substituting Equation 5 into Equation 3 as follows
   F=m/m₀=π[R₀²−R²]LC₀/(πR₀²LC₀)=1−[1−Kt/(C₀R₀)]² (Equation 6)
   dF/dt=2K/C₀R₀−(2K²/C₀²R₀²)t (Equation 7)

Equation 7 indicates that the plot of the rate of active agent released from a cylindrical dosage form without bands versus time will be linear and will decrease with time, as is shown in FIG. 7A. As drug is released from an unbanded capsule, the diameter of the cylinder as well as the area of erosion decreases. In contrast, as the polymeric core of the banded cylinder of this invention shrinks, new surface area is created and exposed to the environment of use (see FIG. 2). As a result, the amount of active agent released over time may remain constant or may increase with time depending on the rate of the new surface area being generated. By arrangement of the number, size and location of bands on the dosage form, the total new surface area created by erosion can be predicted and the desired release profile can be achieved.

The bands may be placed onto the surface of the drug layer such that, as the drug layer erodes, the bands become loose and drop off the drug layer. These bands are easily excreted from the gastrointestinal tract. As the number of bands remaining on the surface of the drug layer decreases, more drug layer surface area will be exposed. The drug layer will therefore erode in a fashion that approaches zero order.

The bands may also be printed onto the surface of the drug layer. The drug layer will erode where not covered by the bands as described above with reference to FIGS. 3-7.

In order to prepare a device of the present invention, the active agent formulation is first prepared and formed into a drug layer of the desired size and shape. In an embodiment, the drug layer in its initial prepared form may be about the size and dimensions of a size “5” to size “000” hard gelatin capsule. The cross-sectional shape of the drug layer may be circular or may be oval, triangular, square, hexagonal or other shapes that are easily handled, especially by patients with limited dexterity. The rings or bands may then placed onto the surface of active agent formulation drug layer or printed onto the surface using conventional banding or printing techniques.

Dosage forms of this invention release effective amounts of active agent to the patient over a prolonged period of time and often provide the opportunity for less frequent dosing, including once-a-day dosing, than previously required for immediate release compositions. The dosage forms of some embodiments of this invention comprise a composition containing a self-dispersing nanoparticle active agent formulation contained in porous particles dispersed in a bioerodible carrier.

Active agents include, inter allia, foods, food supplements, nutrients, drugs, antiacids, vitamins, microorganism attenuators and other agents that provide a benefit in the environment of use. Active agents include any physiologically or pharmacologically active substance that produces a localized or systemic effect or effects in animals, including warm blooded mammals, humans and primates; domestic household or farm animals such as cats, dogs, sheep, goats, cattle, horses and pigs; laboratory animals such as mice, rats and guinea pigs; zoo and wild animals; and the like. Active agents that can be delivered include inorganic and organic compounds, including, without limitation, active agents which act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system and the central nervous system. The terms “active agent” and “drug” are used interchangeably herein. and refer to an agent, drug, compound, composition of matter or mixture thereof which provides some pharmacologic, often beneficial, effect.

Suitable active agents may be selected from, for example, proteins, enzymes, enzyme inhibitors, hormones, polynucleotides, nucleoproteins, polysaccharides, glycoproteins, lipoproteins, polypeptides, steroids, hypnotics and sedatives, psychic energizers, tranquilizers, anticonvulsants, antidepressants, muscle relaxants, antiparkinson agents, analgesics, anti-inflammatories, antihystamines, local anesthetics, muscle contractants, antimicrobials, antimalarials, antivirals, antibiotics, antiobesity agents, hormonal agents including contraceptives, sympathomimetics, polypeptides and proteins capable of eliciting physiological effects, diuretics, lipid regulating agents, antiandrogenic agents, antiparasitics, neoplastics, antineoplastics, antihyperglycemics, hypoglycemics, nutritional agents and supplements, growth supplements, fats, ophthalmics, antienteritis agents, electrolytes and diagnostic agents.

Examples of particular active agents useful in this invention include prochlorperazine edisylate, ferrous sulfate, albuterol, aminocaproic acid, mecamylamine hydrochloride, procainamide hydrochloride, amphetamine sulfate, methamphetamine hydrochloride, benzphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperazine maleate, anisindione, diphenadione erythrityl tetranitrate, digoxin, isoflurophate, acetazolamide, nifedipine, methazolamide, bendroflumethiazide, chlorpropamide, glipizide, glyburide, gliclazide, tobutamide, chlorproamide, tolazamide, acetohexamide, metformin, troglitazone, orlistat, bupropion, nefazodone, tolazamide, chlormadinone acetate, phenaglycodol, allopurinol, aluminum aspirin, methotrexate, acetyl sulfisoxazole, hydrocortisone, hydrocorticosterone acetate, cortisone acetate, dexamethasone and its derivatives such as betamethasone, triamcinolone, methyltestosterone, 17-β-estradiol, ethinyl estradiol, ethinyl estradiol 3-methyl ether, prednisolone, 17-β-hydroxyprogesterone acetate, 19-nor-progesterone, norgestrel, norethindrone, norethisterone, norethiederone, progesterone, norgesterone, norethynodrel, terfandine, fexofenadine, aspirin, acetaminophen, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, selegiline, chlorpromazine, methyldopa, dihydroxyphenylalanine, calcium gluconate, ketoprofen, ibuprofen, cephalexin, erythromycin, haloperidol, zomepirac, ferrous lactate, vincamine, phenoxybenzamine, diltiazem, milrinone, captropril, mandol, quanbenz, hydrochlorothiazide, ranitidine, flurbiprofen, fenbufen, fluprofen, tolmetin, alclofenac, mefenamic, flufenamic, difuninal, nimodipine, nitrendipine, nisoldipine, nicardipine, felodipine, lidoflazine, tiapamil, gallopamil, amlodipine, mioflazine, lisinopril, enalapril, captopril, ramipril, enalaprilat, famotidine, nizatidine, sucralfate, etintidine, tetratolol, minoxidil, chlordiazepoxide, diazepam, amitriptyline, and imipramine, and pharmaceutical salts of these active agents. Further examples are proteins and peptides which include, but are not limited to, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatropin, oxytocin, vasopressin, prolactin, somatostatin, lypressin, pancreozymin, luteinizing hormone, LHRH, interferons, interleukins, growth hormones such as human growth hormone, bovine growth hormone and porcine growth hormone, fertility inhibitors such as the prostaglandins, fertility promoters, growth factors, and human pancreas hormone releasing factor.

It is to be understood that more than one active agent may be incorporated into the active agent formulation in a device of this invention, and that the use of the term “agent” or “drug” in no way excludes the use of two or more such agents or drugs.

The agents can be in various forms, such as uncharged molecules, components of molecular complexes or nonirritating, pharmacologically acceptable salts. Also, simple derivatives of the agents (such as ethers, esters, amides, etc) which are easily hydrolyzed by body pH, enzymes, etc, can be employed.

The present invention has particular utility in the delivery of self-dispersing nanoparticle active agent formulations that are in the form of emulsions or self-emulsifying compositions.. The term emulsion as used in this specification denotes a two-phase system in which one phase is finely dispersed in the other phase. The term emulsifier, as used by this invention, denotes an agent that can reduce and/or eliminate the surface and the interfacial tension in a two-phase system. The emulsifier agent, as used herein, denotes an agent possessing both hydrophilic and lipophilic groups in the emulsifier agent. The term microemulsion, as used herein, denotes a multicomponent system that exhibits a homogenous single phase in which quantities of a drug can be solubilized. Typically, a microemulsion can be recognized and distinguished from ordinary emulsions in that the microemulsion is more stable and usually substantially transparent. The term solution, as used herein, indicates a chemically and physically homogenous mixture of two or more substances.

The emulsion formulations of active agent generally comprise 0.5 wt % to 99 wt % of a surfactant. The surfactant functions to prevent aggregation, reduce interfacial tension between constituents, enhance the free-flow of constituents, and lessen the incidence of constituent retention in the dosage form. The therapeutic emulsion formulations useful in this invention may comprise a surfactant that imparts emulsification comprising a member selected from the group consisting of polyoxyethylenated castor oil comprising 9 moles of ethylene oxide, polyoxyethylenated castor oil comprising 15 moles of ethylene oxide, polyoxyethylene castor oil comprising 20 moles of ethylene oxide, polyoxyethylenated castor oil comprising 25 moles of ethylene oxide, polyoxyethylenated castor oil comprising 40 moles of ethylene oxide, polyoxylenated castor oil comprising 52 moles of ethylene oxide, polyoxyethylenated sorbitan monopalmitate comprising 20 moles of ethylene oxide, polyoxyethylenated sorbitan monolaurate comprising 20 moles of ethylene oxide, polyoxyethylenated sorbitan monooleate comprising 20 moles of ethylene oxide, polyoxyethylenated sorbitan monostearate comprising 20 moles of ethylene oxide, polyoxyethylenated sorbitan monostearate comprising 4 moles of ethylene oxide, polyoxyethylenated sorbitan tristearate comprising 20 moles of ethylene oxide, polyoxyethylenated sorbitan monostearate comprising 20 moles of ethylene oxide, polyoxyethylenated sorbitan trioleate comprising 20 moles of ethylene oxide, polyoxyethylenated stearic acid comprising 8 moles of ethylene oxide, polyoxyethylene lauryl ether, polyoxyethylenated stearic acid comprising 40 moles of ethylene oxide, polyoxyethylenated stearic acid comprising 50 moles of ethylene oxide, polyoxyethylenated stearyl alcohol comprising 2 moles of ethylene oxide, and polyoxyethylenated oleyl alcohol comprising 2 moles of ethylene oxide. The surfactants are available from Atlas Chemical Industries, Wilmington, Del.; Drew Chemical Corp., Boonton, New Jersey; and GAF Corp., New York, N.Y.

Typically, an active agent emulsified formulation useful in the invention initially comprises an oil phase. The oil phase of the emulsion comprises any pharmaceutically acceptable oil which is not miscible with water. The oil can be an edible liquid such as a non-polar ester of an unsaturated fatty acid, derivatives of such esters, or mixtures of such esters can be utilized for this purpose. The oil can be vegetable, mineral, animal or marine in origin. Examples of non-toxic oils comprise a member selected from the group consisting of peanut oil, cottonseed oil, sesame oil, olive oil, corn oil, almond oil, mineral oil, castor oil, coconut oil, palm oil, cocoa butter, safflower, a mixture of mono- and di- glycerides of 16 to 18 carbon atoms, unsaturated fatty acids, fractionated triglycerides derived from coconut oil, fractionated liquid triglycerides derived from short chain 10 to 15 carbon atoms fatty acids, acetylated monoglycerides, acetylated diglycerides, acetylated triglycerides, olein known also as glyceral trioleate, palmitin known as glyceryl tripalmitate, stearin known also as glyceryl tristearate, lauric acid hexylester, oleic acid oleylester, glycolyzed ethoxylated glycerides of natural oils, branched fatty acids with 13 molecules of ethyleneoxide, and oleic acid decylester. The concentration of oil, or oil derivative in the emulsion formulation is 1 wt % to 40 wt %, with the wt % of all constituents in the emulsion preparation equal to 100 wt %. The oils are disclosed in Pharmaceutical Sciences by Remington, 17^{th} Ed., pp. 403-405, (1985) published by Mark Publishing Co., in Encyclopedia of Chemistry, by Van Nostrand Reinhold, 4^{th} Ed., pp. 644-645, (1986) published by Van Nostrand Reinhold Co.; and in U. S. Patent No. 4,259,323 issued to Ranucci.

The dosage form may contain an antioxidant to slow or effectively stop the rate of any autoxidizable material present in the dosage form, particularly if it is in the form of a gelatin capsule. Representative antioxidants comprise a member selected from the group of ascorbic acid; alpha tocopherol; ascorbyl palmitate; ascorbates; isoascorbates; butylated hydroxyanisole; butylated hydroxytoluene; nordihydroguiaretic acid; esters of garlic acid comprising at least 3 carbon atoms comprising a member selected from the group consisting of propyl gallate, octyl gallate, decyl gallate , decyl gallate; 6-ethoxy-2,2,4-trimethyl-1,2-dihydro-guinoline; N-acetyl-2,6-di-t-butyl-p-aminophenol; butyl tyrosine; 3-tertiarybutyl4-hydroxyanisole; 2-tertiary-butyl4-hydroxyanisole; 4-chloro-2,6-ditertiary butyl phenol; 2,6-ditertiary butyl p-methoxy phenol; 2,6-ditertiary butyl-p-cresol: polymeric antioxidants; trihydroxybutyro-phenone physiologically acceptable salts of ascorbic acid, erythorbic acid, and ascorbyl acetate; calcium ascorbate; sodium ascorbate; sodium bisulfite; and the like. The amount of antioxidant used for the present purposes is about 0.001% to 25% of the total weight of the composition present in the dosage form. Antioxidants are known to the prior art in U.S. Pat. Nos. 2,707,154; 3,573,936; 3,637,772; 4,038,434; 4,186,465 and 4,559,237.

The dosage form may also contain a chelating agent to protect the active agent either during storage or when in use. Examples of chelating agents include, for example, polyacrylic acid, citric acid, edetic acid, disodium edetic acid, and the like. The chelating agent may be co-delivered with the active agent in the environment of use to preserve and protect the active agent in situ. Protection is provided for active agents which are inactivated by chelation with multivalent metal cations such as calcium, magnesium or aluminum that may be present in some foods and are at natural background levels in the fluids of the gastrointestinal tract. Such chelating agents may be combined with the self-dispersing nanoparticle active agent formulation in the porous particles, or the chelating agents may be incorporated into the drug layer in which the porous particles are dispersed.

The liquid formulation of the present invention may also comprise a surfactant or a mixture of surfactants where the surfactant is selected from the group consisting of nonionic, anionic and cationic surfactants. Exemplary nontoxic, nonionic surfactants suitable for forming a composition comprise alkylated aryl polyether alcohols known as Triton®; polyethylene glycol tertdodecyl throether available as Nonic®; fatty and amide condensate or Alrosol®; aromatic polyglycol ether condensate or Neutronyx®; fatty acid alkanolamine or Ninol® sorbitan monolaurate or Span®; polyoxyethylene sorbitan esters or Tweens®; sorbitan monolaurate polyoxyethylene or Tween 20®; sorbitan mono-oleate polyoxyethylene or Tween 80®; polyoxypropylene-polyoxyethylene or Pluronic®; polyglycolyzed glycerides such as Labraosol, polyoxyethylated castor oil such as Cremophor and polyoxypropylene-polyoxyethylene-8500 or Pluronic®. By way of example, anionic surfactants comprise sulfonic acids and the salts of sulfonated esters such as sodium lauryl sulfate, sodium sulfoethyl oleate, dioctyl sodium sulfosuccinate, cetyl sulfate sodium, myristyl sulfate sodium; sulated esters; sulfated amides; sulfated alcohols; sulfated ethers; sulfated carboxylic acids; sulfonated aromatic hydrocarbons; sulfonated ethers; and the like. The cationic surface active agents comprise cetyl pyridinium chloride; cetyl trimethyl ammonium bromide; diethylmethyl cetyl ammonium chloride; benzalkonium chloride; benzethonium chloride; primary alkylamonium salts; secondary alkylamonium salts; tertiary alkylamonium salts; quaternary alkylamonium salts; acylated polyamines; salts of heterocyclic amines; palmitoyl carnitine chloride, behentriamonium methosulfate, and the like. Generally, from 0.01 part to 1000 parts by weight of surfactant, per 100 parts of active agent is admixed with the active agent to provide the active agent formulation. Surfactants are known to the prior art in U.S. Pat. No. 2,805,977; and in U.S. Pat. No. 4,182,330.

The liquid formulation may comprise permeation enhancers that facilitate absorption of the active agent in the environment of use. Such enhancers may, for example, open the so-called “tight junctions” in the gastrointestinal tract or modify the effect of cellular components, such a p-glycoprotein and the like. Suitable enhancers include alkali metal salts of salicyclic acid, such as sodium salicylate, caprylic or capric acid, such as sodium caprylate or sodium caprate, and the like. Enhancers may include the bile salts, such as sodium deoxycholate. Various p-glycoprotein modulators are described in U.S. Pat. Nos. 5,112,817 and 5,643,909, which are incorporated herein by reference. Various other absorption enhancing compounds and materials are described in U.S. Pat. No. 5,824,638, which also is incorporated herein by reference. Enhancers may be used either alone or as mixtures in combination with other enhancers.

The self-dispersing nanoparticle active agent formulation of the dosage form may optionally be formulated with inorganic or organic acids or salts of drugs which promote dissolution and disintegration or swelling of the porous particles upon contact with biological fluids. The acids serve to lower the pH of the microenvironment at the porous particle, and promote rapid dissolution of a particle, such as calcium hydrogen phosphate, that is soluble in low pH environments, thus providing rapid liberation of the self-dispersing nanoparticle active agent formulation contained in the porous particle. Examples of organic acids include citric acid, tartaric acid, succinic acid, malic acid, fumaric acid and the like. Salts of drugs where the anion of the salt is acidic, such as acetate, hydrochloride, hydrobromide, sulfate, succinate, citrate, and the like, can be utilized to produce immediate disintegration and dissolution of the porous particle. A more complete list of acidic components for this application is provided in Journal of Pharmaceutical Sciences, “Pharmaceutical Salts”, Review Articles, January, (1977), Vol. 66, No. 1, pages 1-19. The interaction of an acidic component with a porous particle of, for example, calcium hydrogen phosphate, in the presence of water from gastric fluids accelerates dissolution of the particle at a greater rate than gastric fluid alone, producing a more rapid and complete release of the self-dispersing nanoparticle active agent formulation into the environment of use. Likewise alkaline components or salts of drugs where the cation of the salt is alkaline such as choline may be incorporated into the self-dispersing nanoparticle active agent formulation to promote rapid and complete dissolution of a porous particle which is soluble or swells at elevated pH. Such a particle may be formed, for example, of poly(methacrylic acid-methyl methacrylate) 1:2 available commercially as Eudragit S100 (Rohm America, Sommerset, N.J.).

In FIG. 2, a composition is illustrated which contains the porous particles 10 dispersed within a carrier 18. Typically, the composition is compacted as a tablet to form the drug layer portion of the dosage form. During the compacting phase of the manufacture, it is desired that the particle mass 11 be sufficiently non-friable so as to resist pulverization or crushing and undesired exudation of the self-dispersing nanoparticle active agent formulation.

The active compound may be provided in the liquid active agent formulation in amounts of from 1 microgram to 5000 mg per dosage form, depending upon the required dosing level that must be maintained over the delivery period, i.e., the time between consecutive administrations of the dosage forms. More typically, loading of compound in the dosage forms will provide doses of compound to the subject ranging from 1 microgram to 2500 mg per day, more usually 1 mg to 2500 mg per day. The drug layer typically will be a substantially dry composition formed by compression of the carrier and the porous particles, with the understanding that the porous particles will have contained therein the self-dispersing nanoparticle active agent formulation.

The controlled release dosage forms provide a uniform rate of release of compound over a prolonged period of time, typically from about zero hours, the time of administration, to about 4 hours to 20 hours or more, often for 4 hours to 16 hours, and more usually for a time period of 4 hours to 10 hours.

Preparation

Percentages are percentages by weight unless noted otherwise. Variations in the methods and substitution of materials may be made and will be apparent from the earlier description. Equivalent or proportional amounts of such materials may be substituted for those used herein. More specific descriptions are provided in the Examples and alternative materials and procedures are illustrated therein.

Preparation of the Drug Layer

A binder solution is prepared by adding hydroxypropyl cellulose (Klucel MF, Aqualon Company), “HPC”, to water to form a solution containing 5 mg of HPC per 0.995 grams of water. The solution is mixed until the hydroxypropyl cellulose is dissolved. For a particular batch size, a fluid bed granulator (“FBG”) bowl is charged with the required amounts of self-dispersing nanoparticle active agent formulation and the corresponding amount of porous particles, such as exemplified by the calcium hydrogen phosphate particles sold under the trademark FujiCalin. After the liquid is absorbed by the particles, the blend is mixed with, polyethylene oxide (MW 200,000) (Polyox® N-80, Union Carbide Corporation) (20.3%), hydroxypropyl cellulose (Klucel MF) (5%), polyoxyl 40 stearate (3%) and crospovidone (2%). After mixing the semi-dry materials in the bowl, the binder solution prepared as above is added. Then the granulation is dried in the FBG to a dough-like consistency suitable for milling, and the granulation is milled through a 7 or a 10 mesh screen.

The granulation is transferred to a tote blender or a V-blender. The required amounts of antioxidant, butylated hydroxytoluene (“BHT”) (0.01%), and lubricant, stearic acid (1%), are sized through a 40 mesh screen and both are blended into the granulation using the tote or V-blender until uniformly dispersed (about 1 minute of blending for stearic acid and about 10 minutes of blending for BHT.

Assembly of Dosage Form

The granulation may be then compressed into a drug layer having a tablet form. Next, at least two insoluble bands are positioned in spaced relationship on a surface of the drug layer with the surface of the active agent formulation on both sides of each band exposed to the fluid environment of use. Techniques for doing so are disclosed generally in U.S. Pat. Nos. 5,534,263; 5,667,804; and 6,020,000 all to Wong et al.

Assay

The release rate of drug from devices containing the dosage forms of the invention may be determined in standardized assays such as the following. The method involves releasing systems into a release liquid medium, such as acidified water (pH 3), artificial gastric fluid or artificial intestinal fluid. Aliquots of sample release rate solutions are injected onto a chromatographic system to quantify the amount of drug released during specified test intervals. Drug is resolved on a C₁₈ column and detected by UV absorption at the appropriate wavelength for the drug in question. Quantitation is performed by linear regression analysis of peak areas from a standard curve containing at least five standard points.

Samples are prepared with the use of a USP Type 7 Interval Release Apparatus. Each system (invention device) to be tested is weighed. Then, each system is glued to a plastic rod having a sharpened end, and each rod is attached to a release rate dipper arm. Each release rate dipper arm is affixed to an up/down reciprocating shaker (USP Type 7 Interval Release Apparatus), operating at an amplitude of about 3 cm and 2 to 4 seconds per cycle. The rod ends with the attached systems are continually immersed in 50 ml calibrated test tubes containing 50 ml of the release medium, equilibrated in a constant temperature water bath controlled at 37° C.±0.5° C. At the end of each time interval specified, typically one hour or two hours, the systems are transferred to the next row of test tubes containing fresh release medium. The process is repeated for the desired number of intervals until release is complete. Then the solution tubes containing released drug are removed and allowed to cool to room temperature. After cooling, each tube is filled to the 50 ml mark, each of the solutions is mixed thoroughly, and then transferred to sample vials for analysis by high pressure liquid chromatography (“HPLC”). A standard concentration curve is constructed using linear regression analysis. Samples of drug obtained from the release test are analyzed by HPLC and concentration of drug is determined by linear regression analysis. The amount of drug released in each release interval is calculated. Alternatively, concentration of drug may be determined by UV analysis.

Examples 1 and 2, below, illustrate the greater drug loading possible by using nanoparticles of active agent in a drug form having enhanced dissolution characterics. In each example, the same active agent is used and the porous particle carrier, loaded with liquid carrier, performs and can be handled as fine dry granules. In Example 1, the active agent is dissolved into the liquid carrier to its maximum soluble concentration. In Example 2, nanoparticles of the active agent are produced, suspended in the liquid carrier and then loaded into the porous carrier.

The below-described exemplary embodiments are intended to be illustrative in all respects, rather than restrictive, of the present invention. Thus, the present invention is capable of implementation in many variations and modifications that can be derived from the description herein by a person skilled in the art. All such variations and modifications are considered to be within the scope and spirit of the present invention as defined herein.

### V. EXAMPLES

### Example 1

A dosage form, having a total drug layer weight of 500 mg, is formed comprising an active agent that is dissolved in a liquid carrier, a liquid carrier, a porous carrier and other dosage form materials as set out below. In this hypothetical example, the active agent is at its maximum concentration in the liquid carrier at 20 mg of the drug per gram of the liquid carrier.

| | | |
|---|---|---|
| active agent (solubilized in liquid carrier) | 4.4 | mg |
| liquid carrier | 222.8 | mg |
| porous carrier | 222.8 | mg |
| other materials | 50 | mg |
| Total | 500 | mg |

### Example 2

A dosage form, having a total weight of 500 mg, is formed comprising an active agent that is dispersed in a liquid carrier, a liquid carrier, a porous carrier and other dosage form materials (including a push layer) as set out below. The active agent is in the form of nanoparticles, suspended in the liquid carrier and then loaded into the porous carrier.

| | | |
|---|---|---|
| active agent (solubilized in liquid carrier) | 3.6 | mg |
| active agent (in nanoparticle form) | 84.4 | mg |
| liquid carrier | 181.0 | mg |
| porous carrier | 181.0 | mg |
| other materials | 50.0 | mg |
| Total | 500 | mg |

By including the active agent in the drug form as nanoparticles in the liquid carrier, a twenty-fold increase in drug loading in the dosage form is obtained over the dosage form of Example 1. Importantly, also, the dosage form of Example 2, because of the effect of the self-dispersing liquid carrier and the dissolution characteristics of the nano sized particles, still maintains high dissolution characteristics.

Additionally, with self-dispersing nanoparticle formulations loaded into the porous carrier according to the present invention, the self-dispersing nanoparticle formulations can be handled as fine dry particles in the production of dosage forms. The loaded porous carrier can be used to produce solid dosage forms, and, indeed, solid dosage forms that have high drug loading, high dissolution characteristics and high drug bioavailability.

### Example 3

Nanoparticles of megestrol acetate are prepared by making an aqueous suspension of megestrol acetate in 2% Pluronic F108. The suspension is milled for 4 hours on the Dynomill, producing a mean particle size of 0.3 micron. To stabilize the milled drug a polymer solution of hydroxypropyl methylcellulose (HPMC E5) is added to a ratio of Pluronic F108:HPMC E5 1:2. The final milled suspension is then freeze-dried and the resulting nanoparticles have a concentration of 71.2% megestrol acetate.

134 mg of the freeze-dried nanoparticles of megestrol acetate are dispersed into 480 mg of the self-emulsifying liquid carrier (Capric Acid/Cremophor EL, 50/50) and are well mixed to get a suspension of nanoparticles. To convert the suspension into a solid form, 888 mg of Neusilin granules are gradually added into the suspension and mixed well. The final Neusilin/suspension blend produces fine, dry granules. Other excipients, 16 mg Magnesium Stearate and 24 mg Cross Carmellose Sodium (Ac-di-sol), are added to the granules and mixed well. Then, the granules are passed through a 40-mesh screen and tumbled for 30 minutes for further mixing. Finally, the powder is placed in a die cavity having an inside diameter of 9/32inch and compressed with deep concave punch tooling using a pressure head of 2 tons. This forms a longitudinal capsule core. The final 20 mg megestrol acetate tablet weighs 309 mg and has a final composition as listed in the Table 1.

TABLE 1

| Component | Wt % | Mg per dose |
|---|---|---|
| Megestrol | 6.47% | 20.0 |
| Plurnoic F108 | 0.75% | 2.3 |
| HPMC E5 | 1.49% | 4.6 |
| Neusilin | 57.58% | 178.0 |
| Capric Acid | 15.57% | 48.1 |
| Cremophor EL | 15.57% | 48.1 |
| Mg St | 1.03% | 3.2 |
| Acdisol | 1.54% | 4.8 |

### Example 4

The procedure of Example 3 is repeated in this example for providing the following dosage form:

A dosage form, the amount of each component added is identical to that of Example 3, except that the amount of Acdisol added is ⅓ that in Example 3. The final 20 mg megestrol acetate tablet weighs 305 mg.

### Example 5

The procedure of Example 3 is repeated in this example for providing the following dosage form:

A dosage form, the amount of each component added is identical to that of Example 3, except that the amount of Acdisol added is ⅔ of that in Example 3. The final 20 mg megestrol acetate tablet weighs 307 mg.

### Example 6

The procedure of Example 3 is repeated in this example for providing the following dosage form:

A dosage form, the amount of each component added is identical to that of Example 3, except that the amount of Acdisol added is 2 times that of Example 3. The final 20 mg megestrol acetate tablet weighs 313 mg.

### Example 7

The procedure of Example 3 is repeated in this example for providing the following dosage form:

A dosage form, the amount of each component added is identical to that of Example 3, except that the amount of Acdisol added is 3 times that of Example 3. The final 20 mg megestrol acetate tablet weighs 318 mg.

### Example 8

The procedure of Example 3 is repeated to provide the tablet of Example 3. Rings of polyethylene having an inside diameter of 9/32 inch, a wall thickness of 0.013 inch, and a width of 2 mm are then fabricated. These rings, or bands, are press fitted onto the core to complete the dosage form.

### Example 9

The procedure of Example 4 is repeated to provide the tablet of Example 4. Rings of polyethylene having an inside diameter of 9/32 inch, a wall thickness of 0.013 inch, and a width of 2 mm are then fabricated. These rings, or bands, are press fitted onto the core to complete the dosage form.

### Example 10

The procedure of Example 5 is repeated to provide the tablet of Example 5. Rings of polyethylene having an inside diameter of 9/32 inch, a wall thickness of 0.013 inch, and a width of 2 mm are then fabricated. These rings, or bands, are press fitted onto the core to complete the dosage form.

### Example 11

The procedure of Example 6 is repeated to provide the tablet of Example 6. Rings of polyethylene having an inside diameter of 9/32 inch, a wall thickness of 0.013 inch, and a width of 2 mm are then fabricated. These rings, or bands, are press fitted onto the core to complete the dosage form.

### Example 12

The procedure of Example 7 is repeated to provide the tablet of Example 7. Rings of polyethylene having an inside diameter of 9/32 inch, a wall thickness of 0.013 inch, and a width of 2 mm are then fabricated. These rings, or bands, are press fitted onto the core to complete the dosage form.

What is claimed is:

1. A dosage form for the prolonged delivery of a self-dispersing nanoparticle active agent formulation absorbed in porous particles to a fluid environment of use comprising:
a drug layer comprising a self-dispersing nanoparticle active agent formulation absorbed in porous particles, the porous particles being adapted to resist compaction forces sufficient to form a compacted drug layer without significant exudation of the self-dispersing nanoparticle active agent formulation; and
at least two insoluble bands positioned in spaced relationship on a surface of the drug layer with the surface of the active agent formulation on both sides of each band exposed to the fluid environment of use.

2. The dosage form of claim 1, wherein the active agent nanoparticles have a mean particle size of less than about 1500 nm.

3. The dosage form of claim 1 wherein the insoluble bands are between about 0.5 and 8 mm in width.

4. The dosage form of claim 1 wherein the total number of insoluble bands is between 2 and 10.

5. A dosage form for the prolonged delivery of a self-dispersing nanoparticle active agent formulation absorbed in porous particles to a fluid environment of use comprising:
a drug layer comprising a self-dispersing nanoparticle active agent formulation absorbed in porous particles; the porous particles having a mean particle size of ranging from about 50 to about 150 microns and being formed by spray drying a scale-like calcium hydrogen phosphate with a specific surface area of about 20 m²/g to about 60 m²/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:
CaHPO₄.mH₂O
wherein m satisfies the relationship 0≦m≦2.0; and
at least two insoluble bands positioned in spaced relationship on a surface of the drug layer with the surface of the active agent formulation on both sides of each band exposed to the fluid environment of use.

6. The dosage form of claim 5, wherein the active agent nanoparticles have a mean particle size of less than about 1500 nm.

7. The dosage form of claim 5, wherein the insoluble bands are between about 0.5 and 8 mm in width.

8. The dosage form of claim 5, wherein the total number of insoluble bands is between 2 and 10.

9. A dosage form for the prolonged delivery of a self-dispersing nanoparticle active agent formulation absorbed in porous particles to a fluid environment of use comprising:
a drug layer comprising a self-dispersing nanoparticle active agent formulation absorbed in porous particles, the porous particles being calcium hydrogen phosphate having a specific volume of at least 1.5 ml/g, a BET specific surface area of at least 20 m²/g, and a water absorption capacity of at least 0.7 ml/g; and
at least two insoluble bands positioned in spaced relationship on a surface of the drug layer with the surface of the active agent formulation on both sides of each band exposed to the fluid environment of use.

10. The dosage form of claim 9, wherein the active agent nanoparticles have a mean particle size of less than about 1500 nm.

11. The dosage form of claim 9 wherein the insoluble bands are between about 0.5 and 8 mm in width.

12. The dosage form of claim 9 wherein the total number of insoluble bands is between 2 and 10.

13. The dosage form of claim 9, wherein the porous particles have a bulk density of 0.4-0.6 g/ml, a BET surface area of 30-50 m²/g, a specific volume of greater than 2 ml/g, and a mean pore size of at least 50 Angstroms.

14. A dosage form for the prolonged delivery of a self-dispersing nanoparticle active agent formulation absorbed in porous particles to a fluid environment of use comprising:
a drug layer comprising a self-dispersing nanoparticle active agent formulation absorbed in porous particles, the porous particles being calcium hydrogen phosphate having a specific volume of at least 1.5 ml/g, a BET specific area of at least 20 m²/g, and a water absorption capacity of at least 0.7 ml/g, the particles having a size distribution of 100% less than 40 mesh, 50%-100% less than 100 mesh and 10%-60% less than 200 mesh; and
at least two insoluble bands positioned in spaced relationship on a surface of the drug layer with the surface of the active agent formulation on both sides of each band exposed to the fluid environment of use.

15. The dosage form of claim 6, wherein the particles have a size distribution of 100% is less than 40 mesh, 60%-90% is less than 100 mesh and 20%-60% is less than 200 mesh.

16. The dosage form of claim 14 wherein the active agent nanoparticles have a mean particle size of less than about 1500 nm.

17. The dosage form of claim 14, wherein the insoluble bands are between about 0.5 and 8 mm in width.

18. The dosage form of claim 14, wherein the total number of insoluble bands is between 2 and 10.

19. A dosage form for the prolonged delivery of a self-dispersing nanoparticle active agent formulation absorbed in porous particles to a fluid environment of use comprising:
a drug layer comprising a self-dispersing nanoparticle active agent formulation absorbed in porous particles, the porous particles being calcium hydrogen phosphate having a bulk specific volume of 1.5 ml/g-5 ml/g, a BET specific area of 20 m²/g-60 m²/g, a water absorption capacity of at least 0.7 ml/g, and a mean particle size of at least 70 micrometers; and
at least two insoluble bands positioned in spaced relationship on a surface of the drug layer with the surface of the active agent formulation on both sides of each band exposed to the fluid environment of use.

20. The dosage form of claim 19, wherein the active agent nanoparticles have a mean particle size of less than about 1500 nm.

21. The dosage form of claim 19, wherein the insoluble bands are between about 0.5 and 8 mm in width.

22. The dosage form of claim 19, wherein the total number of insoluble bands is between 2 and 10.

23. A method of facilitating the release of an active agent from a dosage form comprising
sorbing a self-dispersing nanoparticle active agent formulation of the active agent into and/or onto a plurality of porous particles, the particles, having a mean particle size of 50-150 microns, being formed by spray drying a scale-like calcium hydrogen phosphate with a specific surface area of 20 m²/g to 60 m²/g, an apparent specific volume of 1.5 ml/g or more, an oil absorption capacity of 0.7 ml/g or more, a primary particle size of 0.1μ to 5μ, and an average particle size of 2μ to 10μ among secondary particles that are aggregates of the primary particles, the scale-like calcium hydrogen phosphate being represented by the following general formula:
CaHPO₄.mH₂O
wherein m satisfies the relationship 0≦m≦2.0; and
placing the self-dispersing nanoparticle active agent formulation of the active agent sorbed into and/or onto a plurality of porous particles into a fluid environment of use, said sorbed plurality of porous particles having at least two insoluble bands positioned in spaced relationship on a surface with the surface on both sides of each band exposed to the fluid environment of use; and
allowing said sorbed plurality of porous particles to erode in the fluid environment of use while the bands remain positioned on the surface;
wherein a surface area of said sorbed plurality of porous particles not covered by the bands increases with time.

24. The method of claim 23, wherein the active agent nanoparticles have a mean particle size of less than about 1500 nm.

25. The method of claim 23, wherein the insoluble bands are between about 0.5 and 8 mm in width.

26. The method of claim 23, wherein the total number of insoluble bands is between 2 and 10.

27. A dosage form for the prolonged delivery of a self-dispersing nanoparticle active agent formulation absorbed in porous particles to a fluid environment of use comprising:
a drug layer comprising a self-dispersing nanoparticle active agent formulation absorbed in porous particles, the porous particles being magnesium aluminometasilicate represented by the general formula
Al₂O₃MgO.2SiO₂.nH₂O
wherein n satisfies the relationship 0≦n≦10; and
at least two insoluble bands positioned in spaced relationship on a surface of the drug layer with the surface of the active agent formulation on both sides of each band exposed to the fluid environment of use.

28. The dosage form of claim 27, wherein the active agent nanoparticles have a mean particle size of less than about 1500 nm.

29. The dosage form of claim 27, wherein the insoluble bands are between about 0.5 and 8 mm in width.

30. The dosage form of claim 27, wherein the total number of insoluble bands is between 2 and 10.

31. A dosage form for the prolonged delivery of a self-dispersing nanoparticle active agent formulation absorbed in porous particles to a fluid environment of use comprising:
a drug layer comprising a self-dispersing nanoparticle active agent formulation absorbed in and/or onto porous particles, the porous particles being magnesium aluminometasilicate represented by the general formula
Al₂O₃MgO.2SiO₂.nH₂O
wherein n satisfies the relationship 0≦n≦10 and having a specific surface area of about 100-300 m²/g, an oil absorption capacity of about 1.3-3.4 ml/g, a mean particle size of about 1-2 microns, an angle of repose about 25°-45°, a specific gravity of about 2 g/ml and a specific volume of about 2.1-12 ml/g; and
at least two insoluble bands positioned in spaced relationship on a surface of the drug layer with the surface of the active agent formulation on both sides of each band exposed to the fluid environment of use.

32. The dosage form of claim 31, wherein the active agent nanoparticles have a mean particle size of less than about 1500 nm.

33. The dosage form of claim 31, wherein the insoluble bands are between about 0.5 and 8 mm in width.

34. The dosage form of claim 31, wherein the total number of insoluble bands is between 2 and 10.
